(19) United States
(12) Patent Application Publication
(10) Pub. No.: US 2026/0216457 A1
(43) Pub. Date: Jul. 30, 2026

(54) ANESTHESIA GAS DELIVERY AND MONITORING SYSTEM

(71) Applicants: H. LEE MOFFITT CANCER CENTER AND RESEARCH INSTITUTE, INC., Tampa, FL (US); INVENTEUR LLC, Lewes, DE (US)

(72) Inventor: Tariq CHAUDHRY, Tampa, FL (US)

(21) Appl. No.: 19/158,214

(22) PCT Filed: Feb. 21, 2024
(86) PCT No.: PCT/US24/16633

§ 371 Date: Aug. 20, 2025

Related U.S. Application Data
(60) Provisional application No. 63/486,084, filed on Feb. 21, 2023.

(51) Int. Cl. A61M 16/01; A61M 16/04; A61M 16/08

(52) U.S. Cl. CPC A61M 16/01; A61M 16/0493; A61M 16/085; A61M 2202/0208; A61M 2202/0241; A61M 2230/432

(57) ABSTRACT

Various implementations include a gas delivery and monitoring apparatus. The apparatus includes a support member, a bite block, a first elongated conduit, a second elongated conduit, and a bridge. The bite block has a lingual side extending between a first and a second side of the bite block. The first elongated conduit defines an exhalation capture flow path terminating in an elongated slot defined by the lingual side of the bite block that extends in a direction from the first side to the second side of the bite block. The second elongated conduit defines a gas delivery flow path terminating in a gas delivery port. Both the gas delivery port and capture inlet are located in the mouth of the subject when the bite block is inserted in the mouth of the subject during use. The bridge extends between the bite block and the support member.

### BACKGROUND

This application claims the benefit of U.S. Provisional Patent Application No. 63/486,084, filed on Feb. 21, 2023, the entire contents of which are incorporated by reference.

### BACKGROUND

Approximately 200,000,000 sedation procedures are performed in the U.S. every year. Patient monitoring during sedation procedures can require the use of a nasal cannula with the prongs inserted into the nostrils to deliver oxygen while monitoring exhaled carbon dioxide. A wide range of issues from inaccurate monitoring to patient injury make this setup unreliable with significant room for improvement.

Nasal insertion of the prongs in patients with oral breathing often leads to a cascade of monitoring issues. Moving the nasal prongs into the patient's mouth is unhygienic but performed regularly in the absence of a better option. Nasal cannulas are insecure and often get dislodged causing loss of carbon dioxide capture as well as inability to deliver oxygen into the nostrils. During facial procedures, nasal cannulas get in the sterile surgical field causing a risk for surgical infections.

Oxygen is heavier than air and tends to settle around the face. Every year several hundred cases of surgical fires causing patient injury are reported when an electrocautery is used. Electrocautery causes sparks and oxygen acts as fuel.

Approximately two million patients in the U.S. are on oxygen therapy either at home or in the nursing homes. Oxygen is delivered through a nasal cannula which is connected to a portable oxygen tank. These patients encounter multiple issues. Nasal cannula tubing has a large surface area that comes in contact with facial skin and long-term use can cause skin irritation, dermatitis or skin ulcers. Continuous oxygen flow through the nostrils can cause mucosal dryness. As prevention, humidifier bottles are added but water vapor condensation not only blocks the oxygen flow to the patient, it can also promote bacterial growth in the tubing. Part of the oxygen delivery through the nasal prongs gets wasted during breath exhalation. A typical oxygen tank lasts 4-5 days. It is estimated that 40-60% of the patients on home oxygen therapy continue to smoke. Lighter flame in close proximity to the plastic prongs emitting oxygen causes serious injuries, death and loss of property each year in the U.S. Therefore, the current nasal cannula lacks appropriate safety and reliability.

### SUMMARY

Various implementations include a gas delivery and monitoring apparatus. The apparatus includes a support member, a bite block, a first elongated conduit, a second elongated conduit, and a bridge. The support member has a longitudinal axis, a proximal end, and a distal end opposite and spaced apart from the proximal end. The bite block is sized to be disposed within a mouth of a subject between at least one tooth of an upper jaw of the subject and at least one tooth of a lower jaw of the subject. The bite block has a proximal end and a distal end opposite and spaced apart from the proximal end. The bite lock has a first side extending between the proximal end and the distal end, a second side opposite and spaced apart from the first side, a lingual side extending between the first side and second side, and a buccal side opposite and spaced apart from the lingual side. The first elongated conduit defines an exhalation capture flow path extending from a capture inlet and terminating in an outlet port. The capture inlet includes an elongated slot defined by the lingual side of the bite block and extends in a direction from the first side to the second side of the bite block. The second elongated conduit defines a gas delivery flow path extending from an inlet port into the bite block and terminating in a gas delivery port such that both the gas delivery port and capture inlet are located in the mouth of the subject when the bite block is inserted in the mouth of the subject during use. The bridge extends between the proximal end of the bite block and the proximal end of the support member.

In some implementations, the capture inlet is a first capture inlet. In some implementations, the first elongated conduit further includes a second capture inlet located at the distal end of the bite block.

In some implementations, the gas delivery port terminates at the distal end of the bite block.

In some implementations, the bridge has a curvature that extends the bridge around a corner of the mouth of the subject when the bite block is disposed within the mouth of the subject between the at least one tooth of the upper jaw of the subject and the at least one tooth of the lower jaw of the subject. In some implementations, when the bite block is disposed within the mouth of the subject between the at least one tooth of the upper jaw of the subject and the at least one tooth of the lower jaw of the subject, the distal end of the support member and the distal end of the bite block are simultaneously positioned on opposite sides of a cheek of the subject and posterior to the corner of the mouth of the subject.

In some implementations, the bridge includes a grip protrusion extending radially outwardly from the curvature. In some implementations, the grip protrusion includes at least one textured surface. In some implementations, the grip protrusion includes a material having a higher friction coefficient than a material of a portion of the bridge from which the grip protrusion extends.

In some implementations, a portion of the first elongated conduit between the bite block and the support member and a portion of the second elongated conduit between the bite block and the support member follows the curvature of the bridge.

In some implementations, the bite block includes a first lingual wing protrusion extending from the first side adjacent the lingual side, a second lingual wing protrusion extending from the second side adjacent the lingual side, a first buccal wing protrusion extending from the first side adjacent the buccal side, and a second buccal wing protrusion extending from the second side adjacent the buccal side. In some implementations, the first buccal wing protrusion extends further from the first side than the first lingual wing protrusion. In some implementations, the second buccal wing protrusion extends further from the second side than the second lingual wing protrusion.

In some implementations, the support member has a channel therewithin extending along the longitudinal axis from a proximal end of the channel to a distal end of the channel. In some implementations, the first elongated extends through the channel from the proximal end to the distal end. In some implementations, the second elongated conduit extends through the channel from the proximal end to the distal end.

In some implementations, the bridge extends away from the support member along a transverse plane perpendicular to the longitudinal axis.

In some implementations, the buccal side of the bite block is spaced apart from the support member by a first minimum distance. In some implementations, the first minimum distance is in the range of 1 to 3 cm. in some implementations, the bridge is deformable such that the buccal side of the bite block is spaced apart from the support member by a second minimum distance. In some implementations, the second minimum distance is in the range of 1 to 3 cm. in some implementations, the bridge provides spring tension to secure the bite block within the mouth of the subject. In some implementations, at least a portion of each of the support member, bite block, and bridge are integrally formed.

### DESCRIPTION OF DRAWINGS

FIG. 1 is a perspective view of a gas delivery and monitoring apparatus according to one implementation.

FIG. 2 is a cutout view of a gas delivery and monitoring apparatus according to one implementation.

FIG. 3 is an illustration of a gas delivery and monitoring apparatus according to one implementation positioned in the mouth of a subject.

FIG. 4 is an illustration of a gas delivery and monitoring apparatus according to one implementation using a “basket” shaped area that can efficiently capture exhaled oral or nasal CO₂.

FIG. 5 is an illustration of a gas delivery and monitoring apparatus according to one implementation using a curved “basket” shaped area that can efficiently capture exhaled oral or nasal CO₂.

FIG. 6 is a perspective view of a gas delivery and monitoring apparatus, according to another implementation.

FIG. 7 is a perspective view of a gas delivery and monitoring apparatus of FIG. 6 disposed in the mouth of a subject.

FIG. 8 is a perspective view of a gas delivery and monitoring apparatus, according to another implementation.

FIG. 9 is a perspective view of a gas delivery and monitoring apparatus of FIG. 8 disposed in the mouth of a subject.

FIG. 10 is a perspective view of a gas delivery and monitoring apparatus, according to another implementation.

FIG. 11A is a perspective view of a gas delivery and monitoring apparatus according to another implementation.

FIG. 11B is a front view of the gas delivery and monitoring apparatus of FIG. 11A disposed within the mouth of a subject.

FIG. 11C is a perspective view of a gas delivery and monitoring apparatus od FIG. 11A.

FIG. 11D is a perspective view of a gas delivery and monitoring apparatus according to another implementation.

FIG. 11E is a front view of the gas delivery and monitoring apparatus of FIG. 11D disposed within the mouth of a subject.

FIG. 12A is a top perspective view of a gas delivery and monitoring apparatus according to another implementation.

FIG. 12B is a bottom perspective view of the gas delivery and monitoring apparatus of FIG. 12A.

FIG. 12C is a top perspective view of the gas delivery and monitoring apparatus of FIG. 12A.

FIG. 12D is a detailed perspective view of the gas delivery and monitoring apparatus of FIG. 12A.

FIG. 12E is a top perspective view of the gas delivery and monitoring apparatus of FIG. 12A.

FIG. 12F is a side view of the gas delivery and monitoring apparatus of FIG. 12A.

### DETAILED DESCRIPTION

The present invention now will be described more fully hereinafter with reference to specific embodiments of the invention. The invention can be embodied in many different forms and should not be construed as limited to the embodiments set forth herein; rather, these embodiments are provided so that this disclosure will satisfy applicable legal requirements.

As used in the specification, and in the appended claims, the singular forms “a,” “an,” “the,” include plural referents unless the context clearly dictates otherwise.

The term “comprising” and variations thereof as used herein are used synonymously with the term “including” and variations thereof and are open, non-limiting terms.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

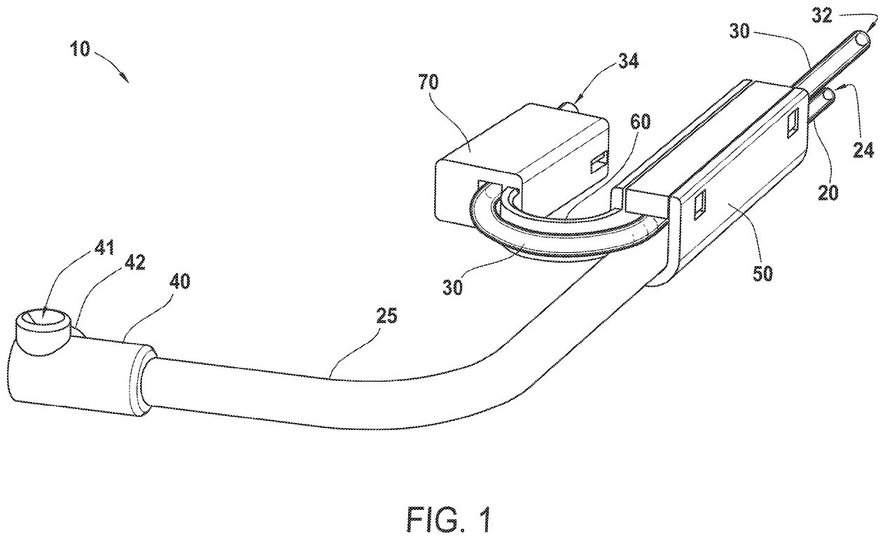

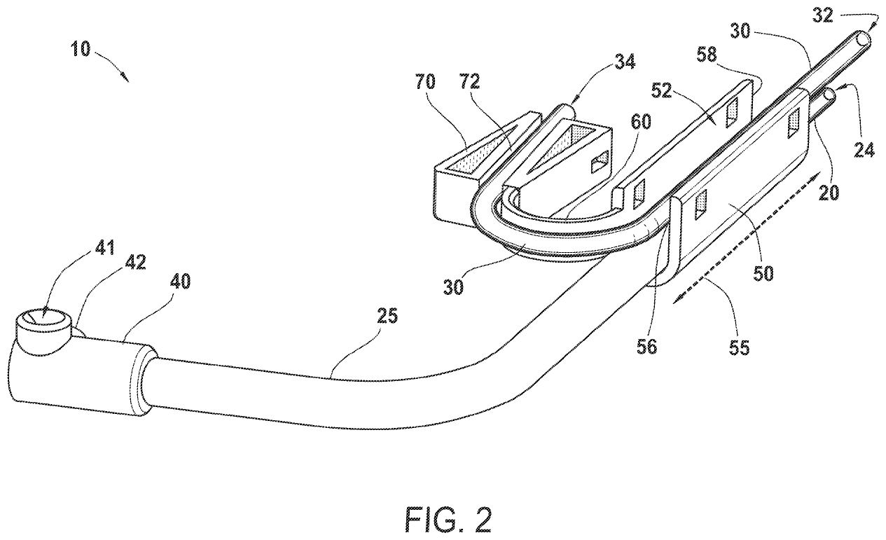

Now referring more particularly to FIGS. 1 and 2 of the drawings, a gas delivery and monitoring apparatus 10 is provided that can be used, for example, to deliver oxygen and monitor exhaled carbon dioxide in a subject while under anesthesia. As shown in FIGS. 1 and 2, the disclosed gas delivery and monitoring apparatus 10 has a first elongated conduit 20 for capturing exhalation from a subject and a second elongated conduit 30 for delivering gas to the subject for inhalation.

The first elongated conduit 20 defines an exhalation capture flow path extending from an exhalation capture manifold 40 to an outlet port 24. In particular embodiments, the exhalation capture manifold 40 captures air exhaled from a subject, which travels through the first elongated conduit to the outlet port 24, where it is connected to a carbon dioxide monitoring system (capnograph). The exhalation capture manifold 40 preferably contains a first capture inlet 41 and a second capture inlet 42 each fluidly connected to the exhalation capture manifold 40. This allows for one inlet to be positioned for capture of exhalation from the nose, while another inlet is positioned for exhalation by the mouth. Therefore, in particular embodiments, the first capture inlet 41 and a second capture inlet 42 have perpendicular flow paths. As shown in FIGS. 1 and 2, the first elongated conduit 20 can be inside a deformable sheath 25, allowing the exhalation capture manifold 40 to be positioned in front of the subject's nose and mouth.

The second elongated conduit 30 defines a gas delivery flow path extending from an inlet port 32 to a gas delivery port 34. In particular embodiments, the inlet port 32 is connected to an oxygen source, which delivers oxygen through the second elongated conduit 30 to the gas delivery port 34. Therefore, in some embodiments of the apparatus, the inlet port 32 is fluidly-connectable to a source of pressurized oxygen.

As shown in FIGS. 1 and 2, the disclosed gas delivery and monitoring apparatus 10 has a support member 50 having a channel 52 therewithin extending along a longitudinal axis 55 from a proximal end 58 of the channel 52 to a distal end 56 of the channel 52. The support member 50 is affixed to a bite block 70 that is sized to be inserted within a mouth of a subject.

As shown in FIG. 2, the first elongated conduit 20 extends from the exhalation capture manifold 40 through the channel 52 from the distal end 56 to the proximal end 58, and terminating in the outlet port 24.

The second elongated conduit 30 extends from the inlet port 32, through the channel 52 from the proximal end 58 to the distal end 56, into the bite block 70, and terminating in the gas delivery port 34. As depicted in FIG. 2, the second elongated conduit 30 can extend through a receiving element 72 (such as a channel) in the bite block. In some cases, the gas delivery port 34 extends past the bite block 70. In other embodiments, the bite block 70 further comprises an outlet manifold fluidly connected to the gas delivery port 34.

The support member 50 and bite block 70 are preferably configured so that they are positioned on opposite sides of a subject's cheek. As depicted in FIGS. 1 and 2, a bridge 60 can connect the bite block 70 to the support member 50. For example, the bridge 60 can extend away from the support member 50 along a transverse plane perpendicular to the longitudinal axis 55, wherein the bridge positions the bite block a first minimum distance from the support element.

This distance is based on the thickness of a subject's cheek. Therefore, in some embodiments, this first minimum distance is about 1 to 3 cm, including about 1.0, 1.5, 2.0, 2.5, or 3.0 cm. The bridge is also preferably deformable to a second minimum distance from the support element to adjust for the size and shape of the subject's mouth and cheek. In these embodiments, the deformable distance (in a direction away from the support member) can be about 0.1 to 2 cm. In some embodiments, deformation of the bridge 60 also provides spring tension to secure the bite block 70 within the mouth of the subject. In some embodiments, at least a portion of each of the support member, bite block, and bridge are integrally formed. In some embodiments, the support member, bite block, and/or bridge are mechanically connected.

The bite block 70 can be a modular embodiment with adjustable heights to accommodate various size mouth openings. The bite block 70 can also have openings/ports to sample oral CO₂.

Each of the first elongated conduit 20 and second elongated conduit 30 can be made from a flexible elastomeric material that is not gas-permeable. Either of these conduits can also be coated or sheathed with another material to provide additional properties, such as rigidity and deformability. In particular embodiments, the first elongated conduit is encased in a deformable sheath 25 that can be articulated. In some embodiments, the sheath 25 is a plastic tube with a co-extruded metal wire that allows the tube to be bent and hold its shape.

Each of the support member 50, bridge 60, and bite block 70 can be made, in whole or in part, from a rigid material, such as a metal or plastic.

The inner diameters of the first and second elongated conduits can be selected based on desired air pressures. For example, in some embodiments, the first elongated conduit has an inner diameter of about 2 to 4 mm. In some embodiments, the second elongated conduit has an inner diameter of about 2 to 8 mm.

The outlet port and inlet port can each independently be any length past the proximal end of the channel, i.e., for connection to a carbon dioxide monitor and oxygen source, respectively. In some cases, the outlet port and/or inlet port are connected to a fitting, such as a luer tube fitting (e.g. male or female). In these cases, the apparatus can be connected to carbon dioxide monitor and oxygen source by extension tubing.

Optionally, the gas delivery and monitoring apparatus 10 can be sterilized, for example by chemical and/or heat based techniques.

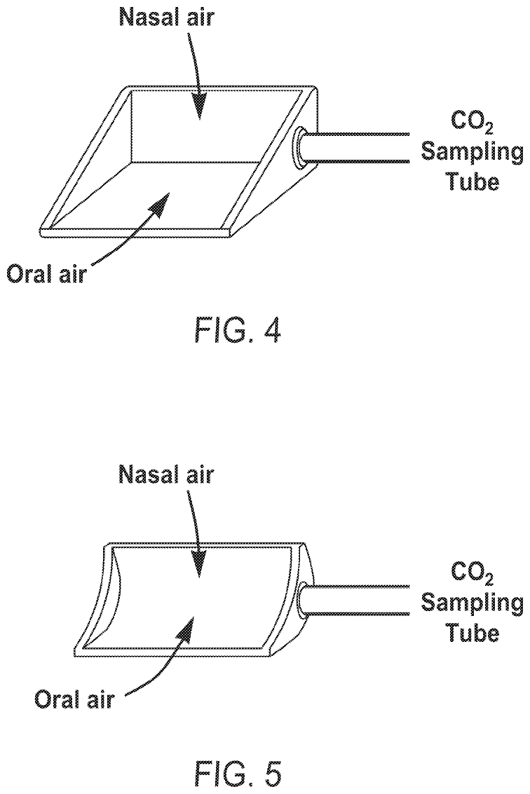

As shown in FIG. 4, in some embodiments, the gas delivery and monitoring apparatus uses a “basket” shaped area that can efficiently capture exhaled oral or nasal CO₂. In some embodiments, the exhalation capture manifold comprises a basket shaped area for the capture of exhaled oral and/or nasal CO₂ though a single capture inlet.

As shown in FIG. 5, in other embodiments, the gas delivery and monitoring apparatus uses a curved “basket” shaped area that can efficiently capture exhaled oral or nasal CO₂. In some embodiments, the exhalation capture manifold comprises a curved basket shaped area for the capture of exhaled oral and/or nasal CO₂ though a single capture inlet. In some embodiments, the single capture inlet is fluidly connected to the exhalation capture manifold. As shown in FIGS. 4 and 5, the first elongated conduit can be connected to the single capture inlet. In some embodiments, the first elongated conduit is located inside a deformable sheath, allowing the exhalation capture manifold to be positioned in front of the subject's nose and mouth.

Sizeable bite blocks can be added to various embodiments described herein to facilitate the placement of an oral-airway, a laryngeal mask airway, or any other life-saving airway apparatus in case of an emergency. Another application of the bite block is to facilitate the passage of an endoscope in the esophagus or trachea of a patient.

In some embodiments, disclosed herein is a method for the delivery of a first gas and monitoring of a second gas, comprising: providing to a subject an apparatus comprising: a support member having a channel therewithin extending along a longitudinal axis from a proximal end of the channel to a distal end of the channel; a bite block affixed to the support member sized to be inserted within a mouth of a subject; a first elongated conduit defining an exhalation capture flow path extending from an exhalation capture manifold, through the channel from the distal end to the proximal end, and terminating in an outlet port; a second elongated conduit defining a gas delivery flow path extending from an inlet port, through the channel from the proximal end to the distal end, into the bite block, and terminating in a gas delivery port; and wherein the exhalation capture manifold captures exhaled oral and/or nasal carbon dioxide. In some embodiments, the first gas comprises oxygen. In some embodiments, the second gas comprises carbon dioxide. In some embodiments, the subject is under anesthesia.

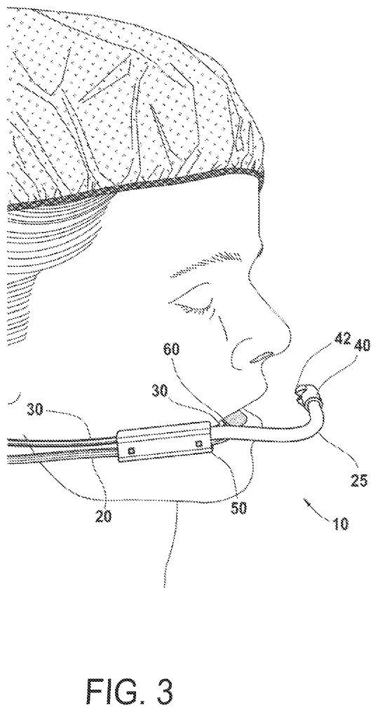

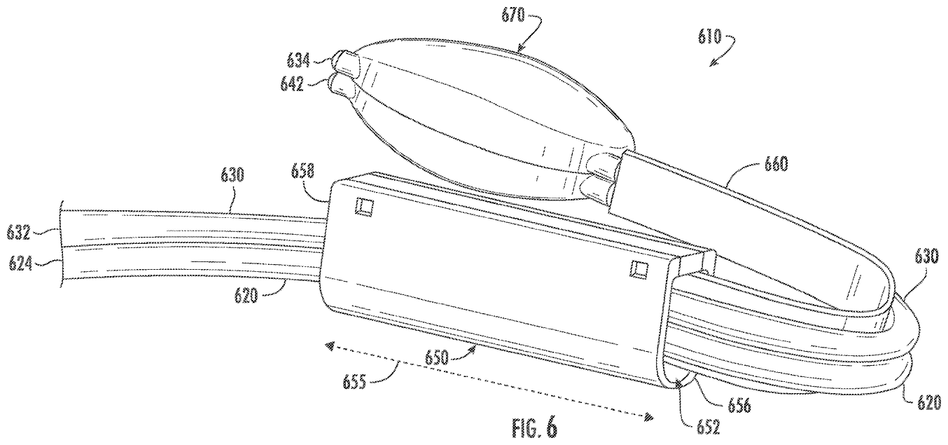

FIGS. 6 and 7 show another embodiment of a gas delivery and monitoring apparatus 610 similar to the gas delivery and monitoring apparatus 10 shown in FIGS. 1-5, but in this embodiment, the first elongated conduit 620 extends through the bite block 670 such that the capture inlet 641 of the first elongated conduit 620 is disposed in the mouth of a subject when the gas delivery and monitoring apparatus 610 is in use. The gas delivery and monitoring apparatus 610 includes a support member 650, a bite block 670, a first elongated conduit 620, a second elongated conduit 630, and a bridge 660. The description of features in the embodiment shown in FIGS. 1-5 can apply to similar features of the embodiment shown in FIGS. 6 and 7.

The support member 650 has a channel 652 therewithin extending along a longitudinal axis 655 from a proximal end 658 of the channel 652 to a distal end 656 of the channel 652. The bridge 660 connects the bite block 670 to the support member 650. The bridge 660 extends away from the support member 650 along a transverse plane perpendicular to the longitudinal axis 655 of the support member 650. The bridge 660 is structured to curve around the corner of the mouth of a subject, and the bite block 670 is sized to be inserted within a mouth of the subject. The support member 650, bite block 670, and bridge 660 shown in FIGS. 6 and 7 are mechanically connected to each other. However, in other embodiments, at least a portion of each of the support member 650, bite block 670, and bridge 660 are integrally formed.

The bridge 660 positions the bite block 670 a first minimum distance from the support member 650 such that the support member 650 and the bite block 670 are simultaneously positionable on opposite sides of the cheek of the subject. The first minimum distance is based on the thickness of a subject's cheek. Therefore, in some embodiments, this first minimum distance is about 1 to 3 cm, including about 1.0, 1.5, 2.0, 2.5, or 3.0 cm. The bridge 660 is also preferably deformable to a second minimum distance from the support member 650 to adjust for the size and shape of the subject's mouth and cheek. In these embodiments, the deformable distance (in a direction away from the support member) can be about 0.1 to 2 cm. In some embodiments, deformation of the bridge 660 also provides spring tension to secure the bite block 670 within the mouth of the subject.

The first elongated conduit 620 defines an exhalation capture flow path. The second elongated conduit 630 defines a gas delivery flow path terminating in an inlet port 632 that is fluidly-connectable to a source of pressurized oxygen. Both the first elongated conduit 620 and the second elongated conduit 630 extend through the channel 652 of the support member 650 similar to the embodiment shown in FIG. 1. However, in the embodiment shown in FIGS. 6 and 7, both the first elongated conduit 620 and the second elongated conduit 630 extend through the bite block 670 such that the capture inlet 641 of the first elongated conduit 620 and the gas delivery port 634 of the second elongated conduit 630 terminate in the mouth of the subject when the gas delivery and monitoring apparatus 610 is in use. To facilitate the first elongated conduit 620 extending through the bite block 670, the bridge 660 is sized such that both the first elongated conduit 620 and the second elongated conduit 630 are extendable along the surface of the bridge 660 to prevent the conduits 620, 630 from kinking when bending around the corner of the mouth of the subject.

Each of the first elongated conduit 620 and second elongated conduit 630 can be made from a flexible elastomeric material that is not gas-permeable. Either of these conduits 620, 630 can also be coated or sheathed with another material to provide additional properties, such as rigidity and deformability. In particular embodiments, the first elongated conduit 620 is encased in a deformable sheath 625 that can be articulated. In some embodiments, the sheath 625 is a plastic tube with a co-extruded metal wire that allows the tube to be bent and hold its shape.

The inner diameters of the first elongated conduit 620 and second elongated conduit 630 can be selected based on desired air pressures. For example, in some embodiments, the first elongated conduit 620 has an inner diameter of about 2 to 4 mm. In some embodiments, the second elongated conduit 630 has an inner diameter of about 2 to 8 mm.

Although FIGS. 6 and 7 show the support member 650 having a channel 652 therewithin, in some embodiments, the support member 650 does not have a channel 652, and the first elongated conduit 620 and the second elongated conduit 630 do not extend through the support member 650.

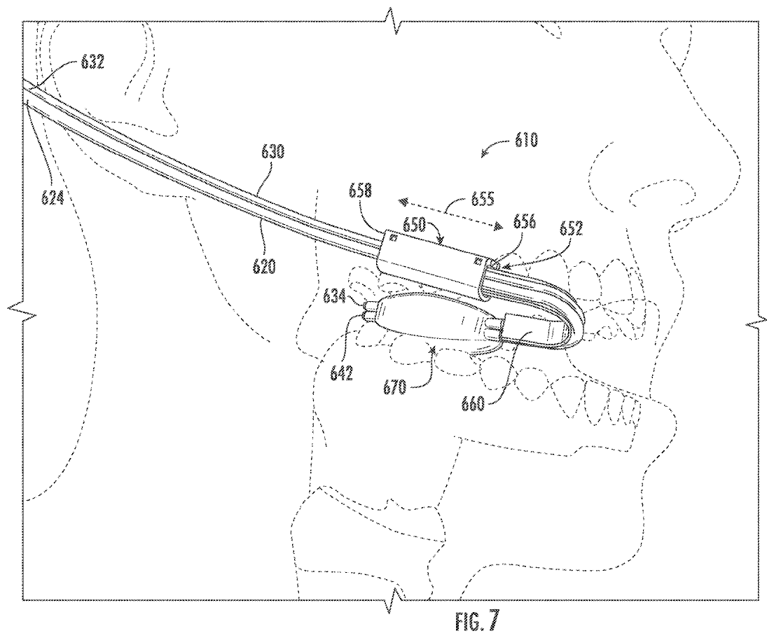

FIGS. 8 and 9 shows another embodiment of a gas delivery and monitoring apparatus 810 similar to the embodiment shown in FIGS. 6 and 7, but in this embodiment, the device does not include a support member or a bridge. Rather, a portion of the first elongated conduit 820 and a portion of the second elongated conduit 830 are rigid and are structured in a U-shape, similar to the shape in which the flexible conduits 620, 630 of the embodiment shown in FIGS. 6 and 7 are held by the support member 650 and the bridge 660. The rigid portions of the first elongated conduit 820 and the second elongated conduit 830 positions the bite block 870 a first minimum distance from the first elongated conduit 820 and the second elongated conduit 830 such that the conduits 820, 830 and the bite block 870 are simultaneously positionable on opposite sides of the cheek of the subject.

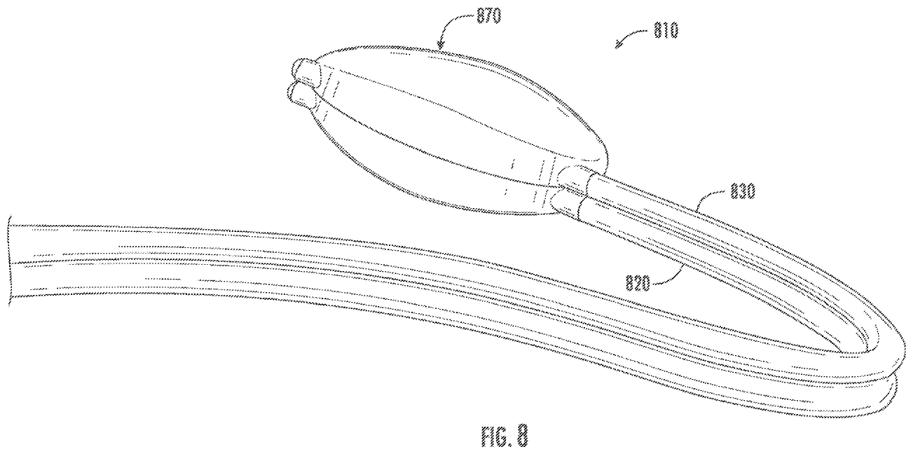

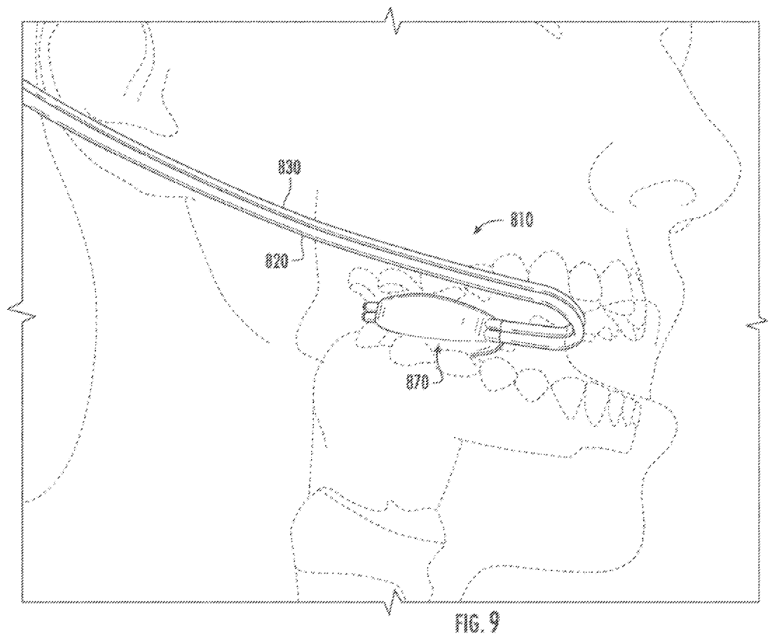

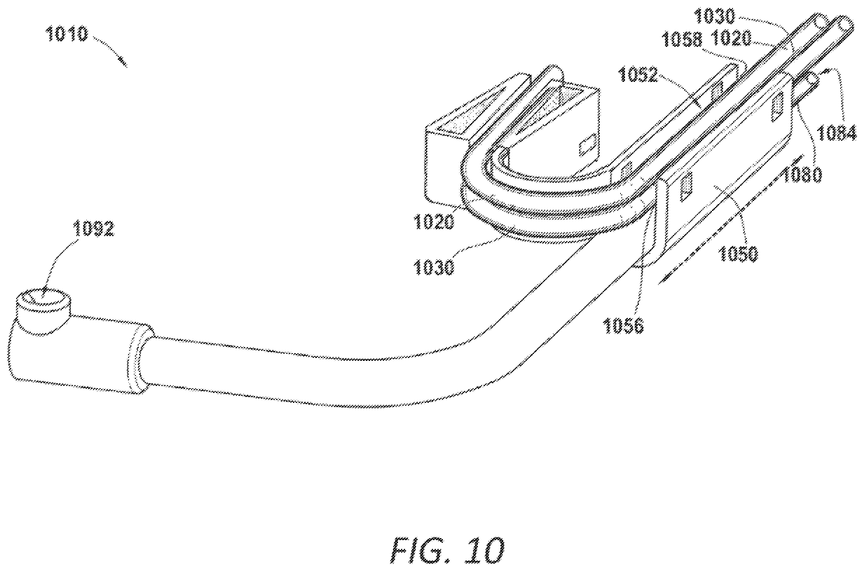

FIG. 10 shows another embodiment of a gas delivery and monitoring apparatus 1010 having a first elongated conduit 1020 and a second elongated conduit 1030 similar to the embodiment shown in FIGS. 6 and 7, but in this embodiment, the device includes a third elongated conduit 1080 for monitoring nasal exhalation. The description of features in the embodiment shown in FIGS. 1-8 can apply to similar features of the embodiment shown in FIG. 10.

The third elongated conduit 1080 is similar to the first elongated conduit 20 of the embodiment shown in FIGS. 1-5. The third elongated conduit 1080 defines a nasal exhalation capture flow path extending from a secondary capture inlet 1092, through the channel 1052 of the support member 1050 from the distal end 1056 to the proximal end 1058, and terminating in a secondary outlet port 1084. The third elongated conduit 1080 extends from the distal end 1056 of the channel 1052 such that a distance between the distal end 1056 of the channel 1052 and the secondary capture inlet 1092 is about 4 to 8 cm.

The third elongated conduit 1080 is made from a flexible elastomeric material that is not gas-permeable and is encased in a deformable sheath. The third elongated conduit 1080 has an inner diameter of 2 to 4 mm, which can be selected based on desired air pressures.

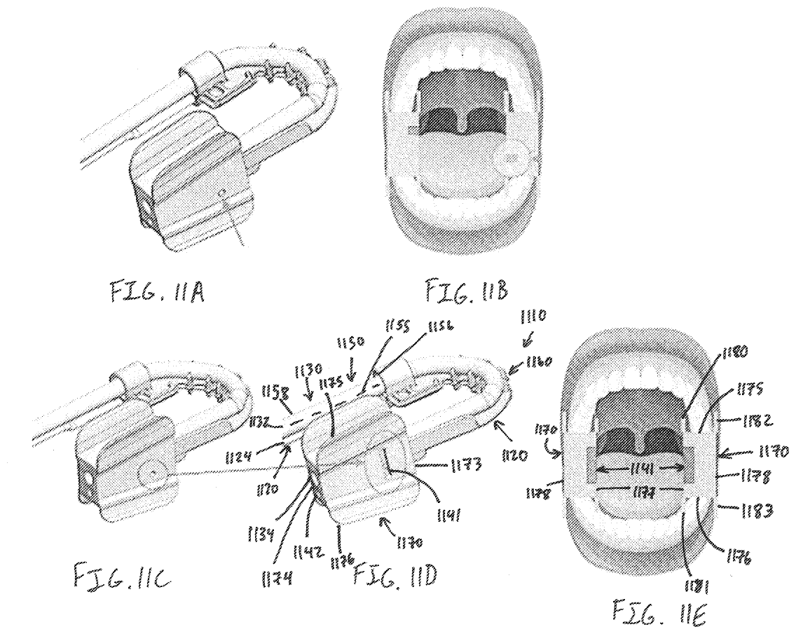

FIGS. 11D and 11E shows a gas delivery and monitoring apparatus 1110, according to one implementation. Although the apparatus 1110 shown in FIGS. 11D and 11E includes specific features, it is contemplated that various other implementations can include any combination of the features of the apparatus 1110 shown in FIGS. 11D and 11E and the features of other apparatuses disclosed herein.

The apparatus 1110 shown in FIGS. 11D and 11E includes a bite block 1170, a first elongated conduit 1120, a second elongated conduit 1130, a support member 1150, and a bridge 1160.

The bite block 1170 shown in FIGS. 11D and 11E has a proximal end 1173, a distal end 1174 opposite and spaced apart from the proximal end 1173, a first side 1175 extending between the proximal end 1173 and the distal end 1174, a second side 1176 opposite and spaced apart from the first side 1175, a lingual side 1177 extending between the first side 1175 and second side 1176, and a buccal side 1178 opposite and spaced apart from the lingual side 1177.

The bite block 1170 is sized to be disposed within the mouth of a subject such that at least one tooth of an upper jaw of the subject and at least one tooth of a lower jaw of the subject contact the first side 1175 and the second side 1176. To aid in retaining the bite block 1170 in this position, the bite block 1170 includes a first lingual wing protrusion 1180 extending from the first side 1175 adjacent the lingual side 1177, a second lingual wing protrusion 1181 extending from the second side 1176 adjacent the lingual side 1177, a first buccal wing protrusion 1182 extending from the first side 1175 adjacent the buccal side 1178, and a second buccal wing protrusion 1183 extending from the second side 1176 adjacent the buccal side 1178. The wing protrusions 1180, 1181, 1182, 1183 are configured to extend on either side of the upper and lower teeth such that the bite block 1170 cannot easily slide lingually or buccally.

As seen in FIGS. 11D and 11E, the first buccal wing protrusion 1182 extends further from the first side 1175 than the first lingual wing protrusion 1180, and the second buccal wing protrusion 1183 extends further from the second side 1176 than the second lingual wing protrusion 1181. The wings on the oral cavity side are shorter than the wings on the cheek side to minimize the chances of the apparatus contacting any instruments placed inside the oral cavity during use. However, in some implementations, any of the wings can extend further or shorter than any other wings for comfort of the subject, for better retention of the device within the mouth of the subject, and/or for reducing the likelihood of contacting instruments in the oral cavity of the subject.

The first elongated conduit 1120 defines an exhalation capture flow path extending from a first capture inlet 1141 and a second capture inlet 1142 to an outlet port 1124. The first capture inlet 1141 includes an elongated slot that is defined by the lingual side 1177 of the bite block 1170. The elongated slot extends in a direction from the first side 1175 to the second side 1176 of the bite block 1170 such that the slot is oriented vertically with respect to the subject when in use (i.e., extends inferior to superior when the bite block 1170 is disposed between upper and lower teeth of the subject). The location of the first capture inlet 1141 on the lingual side 1177 of the bite block 1170 provides for greater exhalation capture, but in many cases, such as the apparatuses shown in FIGS. 11A-11C, the tongue of the subject can become an obstruction to a small opening. By defining the first capture inlet 1141 as a vertically oriented slot, the tongue of the subject is less likely to obstruct the entire length of the elongated slot. Even when the first capture inlet 1141 is partially obstructed, the exhalation of the subject can still be captured through the elongated slot.

The second capture inlet 1142 is located at the distal end 1174 of the bite block 1170, as described above with respect to other implementations. However, in some implementations, the first elongated conduit only includes one capture inlet located either on the lingual side or the distal end of the bite block. In some implementations, one or both of the capture inlets are defined by slots. In some implementations, the bite block defines three or more capture inlets which are each disposed on the same or different sides of the bite block to better capture exhalation from the subject.

The second elongated conduit 1130 defines a gas delivery flow path extending from an inlet port 1132 to a gas delivery port 1134 defined by the bite block 1170. The second elongated conduit terminates in the gas delivery port 1134 such that both the gas delivery port 1134 and capture inlets 1141, 1142 are located in the mouth of the subject when the bite block 1170 is inserted in the mouth of the subject during use. Similar to the second capture inlet 1142, the gas delivery port 1134 terminates at the distal end 1174 of the bite block 1170. This positions the second capture inlet 1142 and the gas delivery port 1134 in the back of the mouth of the subject in use.

The support member 1150 has a longitudinal axis 1155, a proximal end 1158, and a distal end 1156 opposite and spaced apart from the proximal end 1158. The support member 1150 shown in FIGS. 11D and 11E is a rigid portion of the first elongated conduit 1120 and the second elongated conduit 1130. However, in some implementations, the support member includes a body separate from the first elongated conduit and the second elongated conduit, as describe above with respect to other implementations. In such implementations, the support member has a channel therewithin extending along the longitudinal axis from a proximal end of the channel to a distal end of the channel. The first elongated and the second elongated conduit extend through the channel from the proximal end to the distal end.

The bridge 1160 extends between the proximal end 1173 of the bite block 1170 and the proximal end 1158 of the support member 1150. The bridge 1160 extends away from the support member 1150 along a transverse plane perpendicular to the longitudinal axis 1155 of the support member 1150. The bridge 1160 has a curvature that extends the bridge 1160 around a corner of the mouth of the subject when the bite block 1170 is disposed within the mouth of the subject between the at least one tooth of the upper jaw of the subject and the at least one tooth of the lower jaw of the subject. A portion of each of the first elongated conduit 1120 and the second elongated conduit 1130 between the bite block 1170 and the support member 1150 follows the curvature of the bridge 1160. Thus, when the bite block 1170 is disposed within the mouth of the subject between the at least one tooth of the upper jaw of the subject and the at least one tooth of the lower jaw of the subject, the distal end 1156 of the support member 1150 and the distal end 1174 of the bite block 1170 are simultaneously positioned on opposite sides of a cheek of the subject and posterior to the corner of the mouth of the subject.

The bridge 1160 shown in FIGS. 11D and 11E is resiliently flexible and provides spring tension to secure the bite block 1170 within the mouth of the subject. In a resting position, the buccal side 1178 of the bite block 1170 shown in FIGS. 11D and 11E is spaced apart from the support member 1150 by a first minimum distance that is in the range of 1 to 3 cm. The bridge 1160 is deformable to an extended position such that the buccal side 1178 of the bite block 1170 is spaced apart from the support member 1150 by a second minimum distance that is greater than the first minimum distance. In some implementations, the second minimum distance is in the range of 1 to 3 cm.

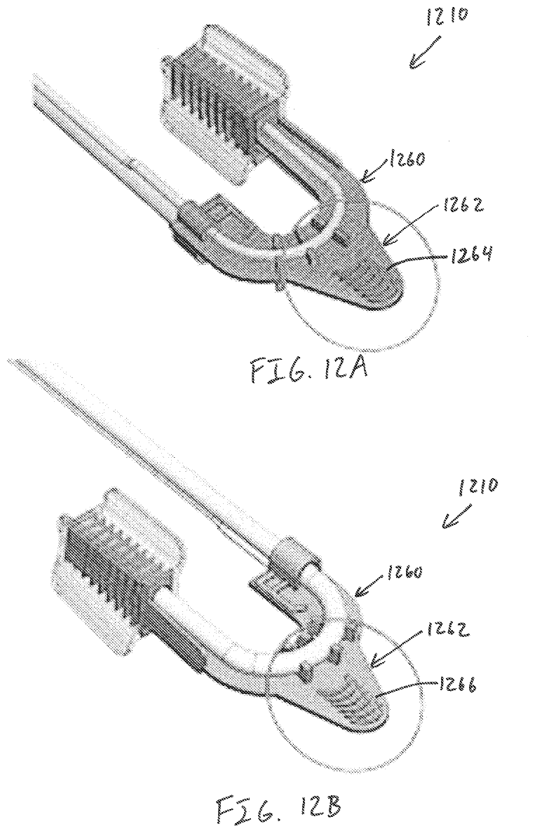

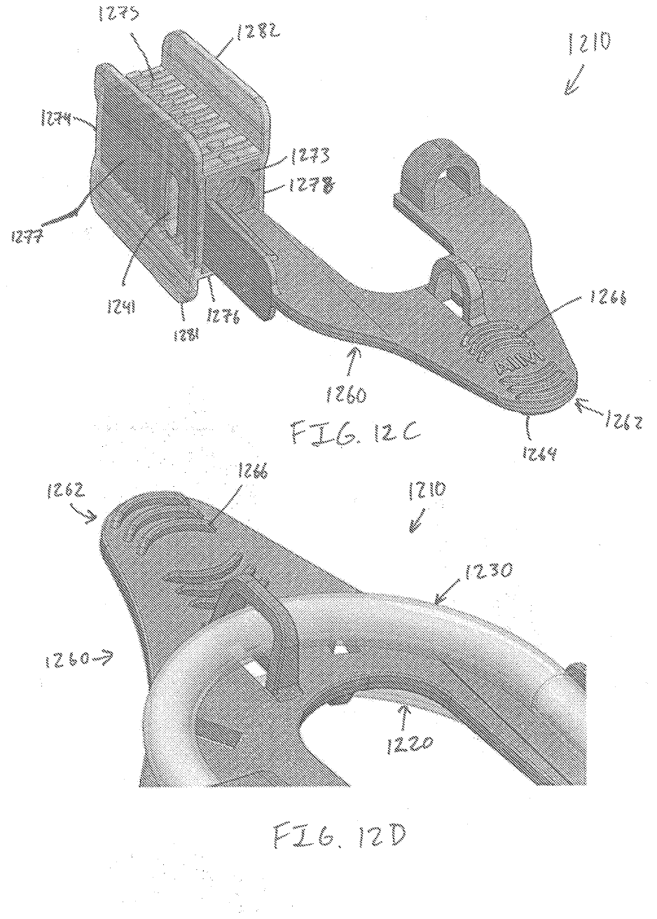

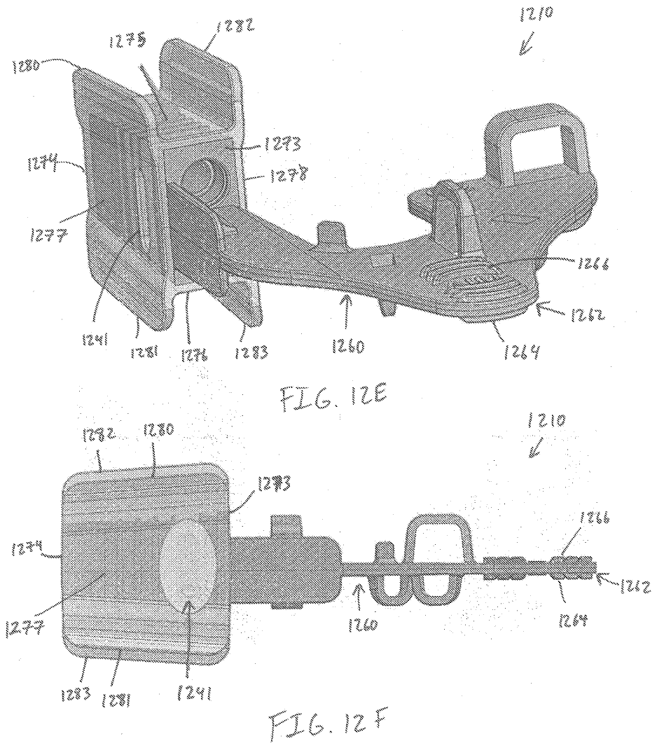

FIGS. 12A-12F shows a gas delivery and monitoring apparatus 1210 according to another implementation. Although the apparatus 1210 shown in FIGS. 12A-12F includes specific features, it is contemplated that various other implementations can include any combination of the features of the apparatus 1210 shown in FIGS. 12A-12F and the features of other apparatuses disclosed herein. The apparatus 1210 of FIGS. 12A-12F includes similar features to the apparatus 1110 of FIGS. 11D and 11F, and therefore, similar reference numbers to those used for the apparatus 1110 of FIGS. 11D and 11F are used to indicate features of the apparatus 1210 of FIGS. 12A-12F. For example, the apparatus shown in FIGS. 12A-12F includes a first elongated conduit 1220 having an outlet port 1224, a first capture inlet 1241, and a second capture inlet 1242, a second elongated conduit 1230 having an inlet port 1232 and a gas delivery port 1234, a support member 1250 having a longitudinal axis 1255, a distal end 1256, and a proximal end 1258, a bridge 1260, and a bite block 1270 having a proximal end 1273, a distal end 1274, a first side 1275, a second end 1276, a lingual side 1277, a buccal side 1278, a first lingual wing protrusion 1280, a second lingual wing protrusion 1281, a first buccal wing protrusion 1282, and a second buccal wing protrusion 1283.

The bridge shown in FIGS. 12A-12F further includes a grip protrusion 1262 extending from a central portion of the bridge 1260. The grip protrusion 1262 extends radially outwardly from the curvature of the bridge 1260 such that the grip protrusion 1262 extends away from the subject in use. The grip protrusion 1262 shown in FIGS. 12A-12F includes a top textured surface 1264 and a bottom textured surface 1266 and includes a coating having a higher friction coefficient than the material of the portion of the bridge 1260 from which the grip protrusion 1262 extends. The grip protrusion 1262 provides a handle for a medical provider wearing surgical gloves to grip an apparatus 1210 that is in a subject's mouth. Although the grip protrusion 1262 shown in FIGS. 12A-12F is a flat tab, in some implementations, the grip protrusion is any other shape that allows the medical provider to more easily grip the apparatus. In some implementations, the grip protrusion includes only one of one or more textured surfaces or a friction coating. In some implementations, the grip protrusion does not include either one or more textured surfaces or a friction coating.

Although the bite block 1170, 1270 and the bridge 1160, 1260 of the apparatuses 1110, 1210 shown in FIGS. 11D-12B are integrally formed, in some implementations, at least a portion of one or more of support member, bite block, and/or bridge are integrally formed. In some implementations, the support member, bite block, and bridge are all separately formed.

A number of embodiments of the invention have been described. Nevertheless, it will be understood that various modifications may be made without departing from the spirit and scope of the invention.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein.

What is claimed is:

1. A gas delivery and monitoring apparatus, comprising:
(a) a support member having a longitudinal axis, a proximal end, and a distal end opposite and spaced apart from the proximal end;
(b) a bite block sized to be disposed within a mouth of a subject between at least one tooth of an upper jaw of the subject and at least one tooth of a lower jaw of the subject, the bite block having a proximal end and a distal end opposite and spaced apart from the proximal end, wherein the bite block has a first side extending between the proximal end and the distal end, a second side opposite and spaced apart from the first side, a lingual side extending between the first side and the second side, and a buccal side opposite and spaced apart from the lingual side,
(c) a first elongated conduit defining an exhalation capture flow path extending from a capture inlet and terminating in an outlet port, wherein the capture inlet includes an elongated slot defined by the lingual side of the bite block and extends in a direction from the first side to the second side of the bite block;
(d) a second elongated conduit defining a gas delivery flow path extending from an inlet port into the bite block, and terminating in a gas delivery port such that both the gas delivery port and the capture inlet are located in the mouth of the subject when the bite block is inserted in the mouth of the subject during use; and
(e) a bridge extending between the proximal end of the bite block and the proximal end of the support member.

2. The apparatus of claim 1, wherein the capture inlet is a first capture inlet, the first elongated conduit further includes a second capture inlet located at the distal end of the bite block.

3. The apparatus of claim 1, wherein the gas delivery port terminates at the distal end of the bite block.

4. The apparatus of claim 1, wherein the bridge has a curvature that extends the bridge around a corner of the mouth of the subject when the bite block is disposed within the mouth of the subject between the at least one tooth of the upper jaw of the subject and the at least one tooth of the lower jaw of the subject.

5. The apparatus of claim 4, wherein, when the bite block is disposed within the mouth of the subject between the at least one tooth of the upper jaw of the subject and the at least one tooth of the lower jaw of the subject, the distal end of the support member and the distal end of the bite block are simultaneously positioned on opposite sides of a cheek of the subject and posterior to the corner of the mouth of the subject.

6. The apparatus of claim 4, wherein the bridge includes a grip protrusion extending radially outwardly from the curvature.

7. The apparatus of claim 6, wherein the grip protrusion includes at least one textured surface.

8. The apparatus of claim 6, wherein the grip protrusion includes a material having a higher friction coefficient than a material of a portion of the bridge from which the grip protrusion extends.

9. The apparatus of claim 1, wherein a portion of the first elongated conduit between the bite block and the support member and a portion of the second elongated conduit between the bite block and the support member follows a curvature of the bridge.

10. The apparatus of claim 1, wherein the bite block includes a first lingual wing protrusion extending from the first side adjacent the lingual side, a second lingual wing protrusion extending from the second side adjacent the lingual side, a first buccal wing protrusion extending from the first side adjacent the buccal side, and a second buccal wing protrusion extending from the second side adjacent the buccal side.

11. The apparatus of claim 10, wherein the first buccal wing protrusion extends further from the first side than the first lingual wing protrusion.

12. The apparatus of claim 10, wherein the second buccal wing protrusion extends further from the second side than the second lingual wing protrusion.

13. The apparatus of claim 1, wherein the support member has a channel therewithin extending along the longitudinal axis from a proximal end of the channel to a distal end of the channel.

14. The apparatus of claim 13, wherein the first elongated extends through the channel from the proximal end to the distal end.

15. The apparatus of claim 13, wherein the second elongated conduit extends through the channel from the proximal end to the distal end.

16. The apparatus of claim 1, wherein the bridge extends away from the support member along a transverse plane perpendicular to the longitudinal axis.

17. The apparatus of claim 1, wherein the buccal side of the bite block is spaced apart from the support member by a first minimum distance, wherein the first minimum distance is 1 cm to 3 cm.

18. The apparatus of claim 1, wherein the bridge is deformable such that the buccal side of the bite block is spaced apart from the support member by a second minimum distance.

19. The apparatus of claim 18, wherein the second minimum distance is 1 cm to 3 cm.

20. The apparatus of claim 18, wherein the bridge provides spring tension to secure the bite block within the mouth of the subject.

21. The apparatus of claim 1, wherein at least a portion of each of the support member, the bite block, and the bridge are integrally formed.
